(19) United States
(12) Patent Application Publication
(10) Pub. No.: US 2021/0141776 A1
(43) Pub. Date: May 13, 2021

(54) PROVIDER COLLABORATION SCORE

(71) Applicant: MARTIN DUNN, ARROYO GRANDE, CA (US)

(72) Inventor: MARTIN DUNN, ARROYO GRANDE, CA (US)

(21) Appl. No.: 16/683,074

(22) Filed: Nov. 13, 2019

(51) Int. Cl. G06F 16/215; G06F 16/23; G16H 10/00

(52) U.S. Cl. CPC G06F 16/215; G16H 10/00; G06F 16/2365

(57) ABSTRACT

An apparatus, system, and method for healthcare provider scoring is described, including a first computing device, the first computing device associated with an owner identifier, a second computing device, a data storage repository, a data records, a first data field, a first data element, a first owner identifier, a first collaboration data set associated with the first owner identifier, the first collaboration data set comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another, and a code segment for determining a collaboration score associated with the owner identifier, wherein the collaboration score is a function of the first collaboration data set.

### BACKGROUND OF THE INVENTION

### 1. Field of the Invention

The present invention relates generally to database data management, and more specifically to database data element quality.

### 2. Discussion of the Related Art

Various systems and processes are known in the art for collecting data from multiple people. In some cases, these techniques are referred to as crowd-sourcing. In one application, crowd sourcing and other data collection techniques may be used to collect health care provider data. However, in some cases, data gathered from a large number of people and/or entities may be difficult to collect and maintain.

For example, a health provider databases may not consistently publish changes given to data elements of healthcare providers in a timely manner, so users (e.g., patients) may be reluctant to reply on this data, because it is not updated by, for example, healthcare providers in a timely fashion. At the same time, there is little incentive for healthcare providers to maintain this data in a timely and accurate way, in part due to an overall shortage in healthcare providers and the large number of heath plans in which these providers may participate. This results in a situation in which providers don't update their data and healthcare provider directories do not timely or accurately publish the updates because few providers update their data in a timely fashion.

When a database of health care information or health care providers becomes outdated, it may cause significant problems ranging from confusion and delay to negative health outcomes.

### SUMMARY

An apparatus, system, and method for healthcare provider scoring are described. The apparatus, system, and method may include a first computing device, the first computing device associated with an owner identifier, a second computing device, a data storage repository, a data records, a first data field, a first data element, a first owner identifier, a first collaboration data set associated with the first owner identifier, the first collaboration data set comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another, and a code segment for determining a collaboration score associated with the owner identifier, wherein the collaboration score is a function of the first collaboration data set.

Another apparatus, system, and method for healthcare provider scoring is described. The apparatus, system, and method may include a computing device, a data storage repository, data records, a first data field, a first data element, an owner identifier, a first collaboration data set associated with the owner identifier, the first collaboration data set comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another, and a code segment for determining a collaboration score associated with the owner identifier, wherein the collaboration score is a function of the first collaboration data set.

Another apparatus, system, and method for healthcare provider scoring is described. The apparatus, system, and method may provide for a first data element, a first data cohort definition, the first data cohort definition comprising a first owner definition, a first informed cohort definition, and first uninformed cohort definition, and a first data item, wherein the first data item is configured to change in response to a first change request from at least one of (a) a first data owner defined by the first owner definition, (b) a plurality of informed requestors defined by the first informed cohort definition numbering in excess of a first informed cohort threshold defined by the first informed cohort definition, and (c) a plurality of uninformed requestors defined by the first uninformed cohort definition numbering in excess of a first uninformed cohort threshold defined by the first uninformed cohort definition.

A method, apparatus, non-transitory computer readable medium, and system for healthcare provider scoring is described. The method, apparatus, non-transitory computer readable medium, and system may include providing a computer system, comprising a number of computing devices with access to a data storage repository comprising data records with distinct data elements, categorizing computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements, modifying a data element in response to a request to modify by either (a) a computing device with an owner identifier as defined by the owner definition, (b) a plurality of informed computing devices as defined by the informed definition, in excess of a threshold of informed computing devices given in the informed definition, and (c) a plurality of uninformed computing devices as defined by the uninformed definition, in excess of a threshold of uninformed computing devices given in the uninformed definition, creating a collaboration data set associated with the identifier of an individual computing device, this data set comprising of at least three of the group of collaboration score factors comprising: response timeous, notification timeous, accuracy, engagement, and contactability, determining the collaboration score associated with the identifier of the computing device as a function of the collaboration data set, wherein each of at least three of the group of factors in the data sets are weighted relative to one another, using a code segment, and determining further a collaboration score associated with all computing devices in the computing system using a code segment.

A method, apparatus, non-transitory computer readable medium, and system for healthcare provider scoring is described. The method, apparatus, non-transitory computer readable medium, and system may include providing an electronic directory for the storing and modifying of data elements, categorizing contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified, modifying said individual data element in the directory in response to a request to modify by either (a) a contributor with an owner identifier as defined by the owner definition, (b) a plurality of informed contributors as defined by the informed definition, in excess of a threshold of informed contributors given in the informed definition, and (c) a plurality of uninformed contributors as defined by the uninformed definition, in excess of a threshold of uninformed contributors given in the uninformed definition, creating a collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another, and determining a collaboration score associated with the contributor using a code segment.

### BRIEF DESCRIPTION OF THE DRAWINGS

FIG. 1 shows an example of a provider relationship network in accordance with aspects of the present disclosure.

FIG. 2 shows an example of a Provider Directory Utility (PDU) provider network in accordance with aspects of the present disclosure.

FIG. 3 shows an example of a provider contracting entanglement in accordance with aspects of the present disclosure.

FIG. 4 shows an example of a hospital privilege at a specific hospital in accordance with aspects of the present disclosure.

FIG. 5 shows an example of an in Health Maintenance Organization (HMO) network with a health plan in accordance with aspects of the present disclosure.

FIG. 6 shows an example of an in Preferred Provider Organization (PPO) network with a health plan in accordance with aspects of the present disclosure.

FIG. 7 shows an example of a physician currently at a specific location in accordance with aspects of the present disclosure.

FIG. 8 shows an example of a healthcare provider scoring system in accordance with aspects of the present disclosure.

FIG. 9 shows an example of a computing device in accordance with aspects of the present disclosure.

FIG. 10 shows an example of an electronic directory in accordance with aspects of the present disclosure.

FIGS. 11 and 12 show examples of a process for determining collaboration scores in accordance with aspects of the present disclosure.

### DETAILED DESCRIPTION

The following description is not to be taken in a limiting sense but is made merely for the purpose of describing the general principles of exemplary embodiments. The scope of the invention should be determined with reference to the claims.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

FIG. 1 shows an example of a provider relationship network 100 in accordance with aspects of the present disclosure. FIG. 1 illustrates how there may be no single point of contact at a service location that can speak for all of a physician's contractual commitments.

Provider relationship network 100 may include at least one government entity 105, one or more health plans 110, one or more group practices 115, one or more Independent Physicians Associations (IPAs) 120, one or more clinics 125, one or more doctors 130, one or more medical facilities 135, one or more medical professionals 140, and a number of patients 145. Each of these elements may be an example of, or include aspects of, the corresponding element or elements described with reference to FIGS. 3-7.

Embodiments of the present disclosure provide a collaborative data exchange application that enables providers (such as doctors 130), provider organizations (including group practice 115, Independent Physicians Association (IPA) 120, clinic 125), and health plans 110 to exchange and verify provider directory data within the provider relationship network 100. Additionally, embodiments include a collaborative platform that leverages subscriber's diligence, accuracy and engagement to maximize value for multiple participants.

A collaborative system has some similarities with “crowd-sourcing” but is not the same. In a collaborative system, certain data has a very specific set of owners which defines the “crowd” from whom the data ought to be collected or validated. For example, a network participation data element may be owned by a provider signatory and health plan signatory. In this case, the informed entities are the contract management staff within the provider organization or health plan 110. The uninformed entities include everyone else (including, for example, other medical professionals 140).

The provider phone number is owned by the provider (i.e., doctor 130), and the informed entities are the authorized staff of the provider, patients 145, administrators, and other people that are in regular contact with the provider. The uninformed entities are everyone else. Handicap accessibility of the medical facility 135 is owned by the facility owner, site auditor, and lessee of the facility. The informed entities are the staff and patients that have visited the facility and the uninformed entities are everyone else.

In a crowd-sourced system, several models have been developed to calculate incentives for contributions. These models may not be suitable in a collaborative environment where a “crowd” is more tightly defined.

Therefore, embodiments of the present disclosure provide a model that measures the behavior of each collaborator as a good citizen of the community. Scores can be used for incentives or sanctions depending on the contractual obligations of the participant.

The score weighs a number of factors such as timeous response to requests (reactive response time), timeous notification of changes (proactive response time), accuracy of data provided (measured against the “master” value as defined in the data policy), engagement in the review of information (clickstream analysis), and contactability (ease of contact).

Similar to a credit score, the weighting of the scoring algorithm may change over time to promote the strategy of the network owner. The end user may not see the scoring algorithm but can be shown the weighting.

As an example, a feature of example embodiments includes a qualified crowd specific to a data element. The qualified crowd gives an opinion by providing input on the information in a data element, e.g., whether the information in the data element is accurate or not; Additionally or alternatively, the opinion may relate to whether the data element includes accurate information.

In some instances, such as medical provider databases, the accuracy of the information can be very low. This may stem from the fact that that doctors 130 in the medical provider databases may have been contracted with multiple different health plans 110 by multiple different contracting entities. Some examples may be: the doctor 130 may sign a contract with his/her multi-specialty practice, the multi-specialty practice may sign a contract with an IPA 120, the independent practitioners association may sign a contract with a managed services organization (MSO), and the managed services organization may sign a contract with a health plan 110.

Consequently, when the doctor 130 determines that he/she would like to close his/her practice, the doctors 130 instruct their front desk staff to begin communicating this information to prospective patients 145 who contact the office to inquire as to whether the doctor 130 can accept them as a new patient 145.

Another solution would be that the doctor 130 could refer to the terms and conditions of their contract with the health plan 110 and, after complying with the termination periods in the contract, the health plan 110 removes the doctor 130 from their directory.

Instead, patients 145 often continue to contact the office based on erroneous information in the health plan's directory. Additionally, in the event the doctor 130 has not properly terminated their relationship with the health plan 110, patients erroneously request information from the doctor's front desk staff. As a result, a very high percentage of doctors 130 listed in the health plan's directory may be unavailable to the patient. However, the directory information may be dependent on the doctor 130 to inform the health plan that the information has changed.

MSO's also may not know that the information has changed. In some cases, the MSO may have been informed that the information has changed. As a result, information flowing from the MSOs to the health plan 110 may be conflicting. However, the MSOs may not know that their information is conflicting. When a conflict is detected, one response could be to ask the owner of the information to confirm if their information is correct. However, the owner may not be responsive to such an inquiry.

The collaboration score of the present disclosure can be used to incentivize owners of the information to be responsive to inquiries to confirm data accuracy, communicate changes in information, provide accurate information, share new information in a timely manner, and be reachable when contacted, among other metrics.

Weights applied to these components of the collaboration score (which can vary by data element) can be adjusted based on predetermined metrics of the directory. For example, if a data element across the directory is inaccurate, compared to missing information, different weightings of different components of the collaboration score can be used to incentivize a doctor 130 to behave in a way that addresses the more heavily weighted component of the directory.

Some of the fields can be changed in response to input from a defined crowd such as a defined informed crowd. For example, if a proportionally large number (e.g., a majority) of MSOs are reporting updated information for the primary phone number of a doctor 130, the primary phone number can be changed. MSOs that have been recently updated may be weighted more heavily than older information from MSO's in determining whether to change the phone number. For example, information changed within four to six weeks may be counted at a greater weight compared to information changed less recently. As such, information changed within the last few weeks may, in effect, get two “votes” to determine whether a “majority” is reporting a change.

Further, a “minority” (for example, ten to twenty percent) of the defined informed crowd may be enough to initiate an inquiry to the owner of the information, such as the doctor 130 or the doctor's office administrator asking the owner to confirm the change. In that case, if the doctor 130 (or administrator) confirms that the information has changed, waiting is optional until a majority of the informed crowd (possibly weighted based on the recency of the information) has confirmed the change. If the owner of the information reports that the information should not be changed, the minority can be informed that the information should not be changed.

If the owner of the information is unresponsive, the information conflict may remain until the majority of the informed crowd has confirmed that the information should be changed such that the MSOs may use different phone numbers until the majority of MSOs have reported that the information should be changed. The definitions of who is the owner, and who is the informed crowd are on a data element basis.

In a variation, an uninformed crowd may also be defined for a data element. There may be a higher threshold for changing information in response to input from the uninformed crowd, and a higher threshold for initiating an inquiry to the owner of the data element based on input from the uninformed crowd.

For example, patients 145 may constitute an uninformed crowd. If a supermajority of patients 145 reports that information on whether a doctor 130 can accommodate Spanish-speaking patients 145 is inaccurate, such information is changed. In a minority (for example twenty to thirty percent) of the defined uninformed crowd, such as patients 145, report that information on whether a doctor 130 can accommodate Spanish-speaking patients 145 is inaccurate, an inquiry can be sent to the owner of the information requesting confirmation as to whether the information is accurate.

In that case, if the doctor 130 (or administrator) confirms that the information has changed, waiting is optional until a supermajority of the uninformed crowd (possibly weighted based on the recency of the information) has confirmed the change. If the owner of the information reports that the information should not be changed, the minority is informed that the information should not be changed. If the owner of the information is unresponsive, the information conflict may remain until the supermajority of the informed crowd has confirmed that the information should be changed.

FIG. 2 shows an example of a Provider Directory Utility (PDU) provider network 200 in accordance with aspects of the present disclosure. A PDU provider network 200 may include individual physicians 205, health plans 210, medical groups 215, industry associations 220, reference data 225, participating provider groups 230, hospitals and clinics 235, and group practices 240.

A PDU provider network may ensure collaboration between peers, protect data privacy, reduce administration costs for all parties, collect data from the proper source, and connect operational systems. As described above with reference to FIG. 1, some examples of the present disclosure include providing a collaboration score for various participants to ensure that information is accurately collected and maintained.

FIG. 3 shows an example of a provider contracting entanglement 300 in accordance with aspects of the present disclosure. Provider contracting entanglement 300 illustrates an example of the relationships between different entities in a health care system, and the flow of information between these entities.

Provider contracting entanglement 300 may include HMO health plan 305, PPO health plan 306, Accountable Care Organization (ACO) 310, IPA 315, Management Services Organizations (MSO) 320, medical facility 325, group practice 330, doctor 335, and patients 340. Each of these elements may be an example of, or include aspects of, the corresponding element or elements described with reference to FIGS. 1, and 4-7.

FIG. 4 shows an example of a hospital privilege at a specific hospital in accordance with aspects of the present disclosure. The entities comprising a primary source, qualified crowd, and unqualified crowd are noted in the key.

Provider contracting entanglement 400 may be an example of, or include aspects of, the corresponding element or elements described with reference to FIGS. 3, and 5-7 and may include HMO health plan 405, PP health plan 406, ACO 410, IPA 415, MSO 420, medical facility 425, group practice 430, doctor 435, and patients 440. Each of these elements may be an example of, or include aspects of, the corresponding element or elements described with reference to FIGS. 1, 3, and 5-7.

Here, the doctor 435 and the medical facility 425 are primary sources, while the PPO health plan 406, IPA 415, and MSOs 420 are part of the qualified crowd. The HMO health plan 405, ACO 410, group practice 430, and patients 440 are unqualified.

FIG. 5 shows an example of an in-Health Maintenance Organization (HMO) network with an HMO health plan 505 in accordance with aspects of the present disclosure. Here, the primary source, qualified crowd, and unqualified crowd are noted in the key.

Provider contracting entanglement 500 may be an example of, or include aspects of, the corresponding element or elements described with reference to FIGS. 3, 4, 6, and 7 and may include HMO health plan 505, PPO health plan 506, ACO 510, IPA 515, MSO 520, medical facility 525, group practice 530, doctor 535, and patients 540. Each of these elements may be an example of, or include aspects of, the corresponding element or elements described with reference to FIGS. 1, 3, 4, 6, and 7.

The HMO health plan 505 and IPA 515 are primary sources, while the doctors 535 and group practice 530 are qualified crowds. The PPO health plan 506, ACO 510, MSO 520, and the patients 540 are part of the unqualified crowd.

FIG. 6 shows an example of an in-Preferred Provider Organization (PPO) network with a health plan 605 in accordance with aspects of the present disclosure. The primary source, qualified crowd, and unqualified crowd are noted in the key.

Provider contracting entanglement 600 may be an example of, or include aspects of, the corresponding element or elements described with reference to FIGS. 3-5, and 7 and may include HMO health plan 605, PPO health plan 606, ACO 610, IPA 615, MSO 620, medical facility 625, group practice 630, doctor 635, and patients 640. Each of these elements may be an example of, or include aspects of, the corresponding element or elements described with reference to FIGS. 1, 3-5, and 7.

The PPO health plan 606 and the doctors 635 are primary sources, while the HMO health plan 605, ACO 610, IPA 615, MSO 620, group practice 630, and patients 640 represent an unqualified crowd.

FIG. 7 shows an example of a physician is currently at a specific location in accordance with aspects of the present disclosure. The primary source, qualified crowd, and unqualified crowd are noted in the key.

Provider contracting entanglement 700 may be an example of, or include aspects of, the corresponding element or elements described with reference to FIGS. 3-6 and may include HMO health plan 705, PPO health plan 706, ACO 710, IPA 715, MSO 720, medical facility 725, group practice 730, doctor 735, and patients 740. Each of these elements may be an example of, or include aspects of, the corresponding element or elements described with reference to FIGS. 1, and 3-6.

The doctors 735 and group practice 730 are primary sources, while the HMO health plan 705, PPO health plan 706, ACO 710, IPA 715, MSO 720 represent a qualified crowd. The patients 740 are the unqualified crowd.

FIG. 8 shows an example of a healthcare provider scoring system 800 in accordance with aspects of the present disclosure. Healthcare provider scoring system 800 may be applied to a provider relationship network 100 as described with reference to FIG. 1, in the context of a provider contracting entanglement as described with reference to FIGS. 3-7.

Healthcare provider scoring system 800 may include first computing device 805, second computing device 810, and network 815. First computing device 805 and second computing device 810 may be examples of computing devices operated by users in a provider relationship network 100. Two computing devices are shown as an example, but more computing devices may be a part of a provider relationship network 100.

First computing device 805 may be associated with an owner identifier. In some cases, First computing device 805 or second computing device 810 may be categorized by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements.

In another example, first computing device 805 or second computing device 810 may be categorized by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified.

In another example, first computing device 805 or second computing device 810 may be categorized by a first contributor to a data element by an identifier tag, possible identifiers consisting of (a) an owner identifier, (b) an informed identifier, and (c) a uninformed identifier, with each of said identifiers being attributed to a first contributor by (a) a first owner identifier definition, (b) a first informed identifier definition, and (c) a first uninformed identifier definition, with each of the definitions being unique to the data element being modified.

FIG. 9 shows an example of a computing device 900 in accordance with aspects of the present disclosure. Computing device 900 may be an example of first computing device 805 or second computing device 810 described above with reference to FIG. 8.

A computing device 900 may be a personal computer, laptop computer, mainframe computer, palmtop computer, personal assistant, mobile device, or any other suitable processing apparatus. Computing device 900 may include data storage repository 905 and code segment 935.

In some examples, data storage repository 905 may be a part of a computer system that includes a number of computing devices 900 with access to the data storage repository 905, including data records 910 and data elements 920.

Data storage repository 905 may provide an electronic directory for the storing and modifying of data elements 920. Data storage repository 905 may include data records 910.

Data records 910 may include one or more data fields 915. In some examples, a first data field 915 does not include a particular data set (i.e., a seventh collaboration data set 930 associated with the uninformed user identifier).

Data field 915 may include data element 920, owner identifier 925, and collaboration data set 930.

Data element 920 may be modified in response to a request to modify by either (a) a computing device 900 with an owner identifier 925 as defined by the owner definition, (b) a set of informed computing devices 900 as defined by the informed definition, in excess of a threshold of informed computing devices 900 given in the informed definition, and (c) a set of uninformed computing devices 900 as defined by the uninformed definition, in excess of a threshold of uninformed computing devices 900 given in the uninformed definition.

Data element 920 may be modified response to a request to modify by either (a) a contributor with an owner identifier 925 as defined by the owner definition, (b) a set of informed contributors as defined by the informed definition, in excess of a threshold of informed contributors given in the informed definition, and (c) a set of uninformed contributors as defined by the uninformed definition, in excess of a threshold of uninformed contributors given in the uninformed definition. Data element 920 may also be modified in response to a request to modify by either (a) a first contributor with an owner identifier 925 as defined by the owner definition, (b) a set of informed contributors as defined by the first informed definition, in excess of a threshold of informed contributors given in the first informed definition, and (c) a set of uninformed contributors as defined by the first uninformed definition, in excess of a threshold of first uninformed contributors given in the uninformed definition. Data element 920 may also be modified in response to a request to modify by either (a) a second contributor with an owner identifier 925 as defined by the owner definition, (b) a set of informed contributors as defined by the second informed definition, in excess of a threshold of informed contributors given in the second informed definition, and (c) a set of uninformed contributors as defined by the second uninformed definition, in excess of a threshold of second uninformed contributors given in the uninformed definition.

Data element 920 may be an example of, or include aspects of, the corresponding element or elements described with reference to FIG. 10.

In some examples, said first owner identifier 925 identifies a first owner. In some examples, said second owner identifier 925 identifies a second owner. In other words, different data elements 920 may be associated with different owners.

One or more collaboration data sets 930 may be associated with the owner identifiers. For example, a first collaboration data set 930 may be associated with a first owner identifier 925, the first collaboration data set 930 comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another.

The first collaboration data set 930 may be associated with the owner identifier 925, the first collaboration data set 930 comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another.

A second collaboration data set 930 may also be associated with the second owner identifier 925, the second collaboration data set 930 comprising a second collaboration score being determined as a function of a second at least three of the group of collaboration score factors comprising: response timeous, notification timeous, accuracy, engagement, and contactability, wherein each of the second at least three of the group of collaboration score factors is weighted relative to one another.

The second collaboration data set 930 may also be associated with the owner identifier 925, the second collaboration data set 930 comprising a second collaboration score being determined as a function of a second at least three of the group of collaboration score factors comprising: response timeous, notification timeous, accuracy, engagement, and contactability, wherein each of the second at least three of the group of collaboration score factors is weighted relative to one another.

A third collaboration data set 930 may also be associated with the first informed user identifier, the third collaboration data set 930 comprising a third collaboration score being determined as a function of a third at least three of the group of collaboration score factors comprising: response timeous, notification timeous, accuracy, engagement, and contactability, wherein each of the third at least three of the group of collaboration score factors is weighted relative to one another.

A fourth collaboration data set 930 may also be associated with the first informed user identifier, the fourth collaboration data set 930 comprising a fourth collaboration score being determined as a function of a fourth at least three of the group of collaboration score factors comprising: response timeous, notification timeous, accuracy, engagement, and contactability, wherein each of the fourth at least three of the group of collaboration score factors is weighted relative to one another.

A fifth collaboration data set 930 may also be associated with the second informed user identifier, the fifth collaboration data set 930 comprising a fifth collaboration score being determined as a function of a fifth at least three of the group of collaboration score factors comprising: response timeous, notification timeous, accuracy, engagement, and contactability, wherein each of the fifth at least three of the group of collaboration score factors is weighted relative to one another.

A sixth collaboration data set 930 may also be associated with the second informed user identifier, the sixth collaboration data set 930 comprising a sixth collaboration score being determined as a function of a sixth at least three of the group of collaboration score factors comprising: response timeous, notification timeous, accuracy, engagement, and contactability, wherein each of the sixth at least three of the group of collaboration score factors is weighted relative to one another.

A collaboration data set 930 may be associated with the identifier of an individual computing device 900, this data set including at least three of the group of collaboration score factors including: response timeous, notification timeous, accuracy, engagement, and contactability.

A collaboration data set 930 may be associated with the contributor, this data set including a collaboration score determined as a function of at least three of the group of collaboration score factors including: response timeous, notification timeous, accuracy, engagement, and contactability, where each of at least three of the group of factors is weighted relative to one another.

A collaboration data set 930 may also be associated with the contributor, this data set including a collaboration score determined as a function of at least three of the group of collaboration score factors including: response timeous, notification timeous, accuracy, engagement, and contactability, where each of at least three of the group of factors is weighted relative to one another.

A collaboration data set 930 may be associated with the contributor, this data set including a collaboration score determined as a function of at least three of the group of collaboration score factors including: response timeous, notification timeous, accuracy, engagement, and contactability, where each of at least three of the group of factors is weighted relative to one another.

Code segment 935 may determine a collaboration score associated with the owner identifier 925, wherein the collaboration score is a function of the first collaboration data set 930. In some examples, said code segment 935 is for determining said collaboration score associated with said owner identifier 925, where said collaboration score is a function of said first collaboration data set 930 and the second collaboration data set 930.

In some examples, said first collaboration score is determined as a function of an age of each of the first at least three of the group of collaboration score factors. In some examples, said second collaboration score is determined as a function of an age of each of the second at least three of the group of collaboration score factors. In some examples, said third collaboration score is determined as a function of an age of each of the third at least three of the group of collaboration score factors. In some examples, said fourth collaboration score is determined as a function of an age of each of the fourth at least three of the group of collaboration score factors. In some examples, said fifth collaboration score is determined as a function of an age of each of the fifth at least three of the group of collaboration score factors.

In some examples, said first collaboration score is determined as a function of an age of each of the first at least three of the group of collaboration score factors, where older ones of the first at least three of the group of collaboration score factors are weighted less than newer ones of the first at least three of the group of collaboration score factors. In some examples, said second collaboration score is determined as a function of an age of each of the second at least three of the group of collaboration score factors, where older ones of the second at least three of the group of collaboration score factors are weighted less than newer ones of the second at least three of the group of collaboration score factors.

In some examples, said third collaboration score is determined as a function of an age of each of the third at least three of the group of collaboration score factors, where older ones of the third at least three of the group of collaboration score factors are weighted less than newer ones of the third at least three of the group of collaboration score factors. In some examples, said fourth collaboration score is determined as a function of an age of each of the fourth at least three of the group of collaboration score factors, where older ones of the fourth at least three of the group of collaboration score factors are weighted less than newer ones of the fourth at least three of the group of collaboration score factors.

In some examples, said fifth collaboration score is determined as a function of an age of each of the fifth at least three of the group of collaboration score factors, where older ones of the fifth at least three of the group of collaboration score factors are weighted less than newer ones of the fifth at least three of the group of collaboration score factors.

Code segment 935 may determine a collaboration score associated with the owner identifier 925, wherein the collaboration score is a function of the first collaboration data set 930. In some examples, said code segment 935 for determining said collaboration score associated with said owner identifier 925, where said collaboration score is a function of said first collaboration data set 930 and the second collaboration data set 930.

Code segment 935 may determine the collaboration score associated with the identifier of the computing device 900 as a function of the collaboration data set 930, where each of at least three of the group of factors in the data sets are weighted relative to one another, using a code segment 935. Code segment 935 may also determine further a collaboration score associated with all computing devices 900 in the computing system using a code segment 935.

Code segment 935 may determine a collaboration score associated with the contributor using a code segment 935. Code segment 935 may also determine a first collaboration score associated with the contributor using a code segment 935. Code segment 935 may also determine a collaboration score associated with the contributor using a code segment 935, where the collaboration score is a function of the first collaboration data set 930 and the second collaboration data set 930.

FIG. 10 shows an example of an electronic directory 1000 in accordance with aspects of the present disclosure. Electronic directory 1000 may include data element 1005. Data element 1005 may include data cohort 1010 and data item 1015. Data element 1005 may be an example of, or include aspects of, the corresponding element or elements described with reference to FIG. 9.

Data cohort 1010 may be defined according to a data cohort definition. A first data cohort definition may comprise a first owner definition, a first informed cohort definition, and first uninformed cohort definition. A second data cohort definition may comprise a second owner definition, a second informed cohort definition, and a second uninformed cohort definition.

Data item 1015 may be configured to change in response to a first change request from at least one of (a) a first data owner defined by the first owner definition, (b) a plurality of informed requestors defined by the first informed cohort definition numbering in excess of a first informed cohort threshold defined by the first informed cohort definition, and (c) a plurality of uninformed requestors defined by the first uninformed cohort definition numbering in excess of a first uninformed cohort threshold defined by the first uninformed cohort definition. Data item 1015 may also be configured to change in response to a second change request from at least one of (a) a second data owner defined by the second owner definition, (b) a plurality of informed requestors defined by the second informed cohort definition numbering in excess of a second informed cohort threshold defined by the second informed cohort definition, and (c) a plurality of uninformed requestors defined by the second uninformed cohort definition numbering in excess of a second uninformed cohort threshold defined by the second uninformed cohort definition.

FIG. 11 shows an example of a process for determining collaboration scores in accordance with aspects of the present disclosure. In some examples, these operations may be performed by a system including a processor executing a set of codes to control functional elements of an apparatus. Additionally or alternatively, the processes may be performed using special-purpose hardware. Generally, these operations may be performed according to the methods and processes described in accordance with aspects of the present disclosure. For example, the operations may be composed of various substeps, or may be performed in conjunction with other operations described herein.

At step 1100, a computer system is provided, including a number of computing devices with access to a data storage repository including data records with distinct data elements. In some cases, the operations of this step may refer to, or be performed by, a data storage repository as described with reference to FIG. 9.

At step 1105, the system categorizes computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements. In some cases, the operations of this step may refer to, or be performed by, a first computing device as described with reference to FIG. 8.

At step 1110, the system modifies a data element in response to a request to modify by either (a) a computing device with an owner identifier as defined by the owner definition, (b) a set of informed computing devices as defined by the informed definition, in excess of a threshold of informed computing devices given in the informed definition, and (c) a set of uninformed computing devices as defined by the uninformed definition, in excess of a threshold of uninformed computing devices given in the uninformed definition. In some cases, the operations of this step may refer to, or be performed by, a data element as described with reference to FIGS. 9 and 10.

At step 1115, the system creates a collaboration data set associated with the identifier of an individual computing device, this data set including of at least three of the group of collaboration score factors including: response timeous, notification timeous, accuracy, engagement, and contactability. In some cases, the operations of this step may refer to, or be performed by, a collaboration data set as described with reference to FIG. 9.

At step 1120, the system determines the collaboration score associated with the identifier of the computing device as a function of the collaboration data set, where each of at least three of the group of factors in the data sets are weighted relative to one another, using a code segment. In some cases, the operations of this step may refer to, or be performed by, a code segment as described with reference to FIG. 9.

At step 1125, the system further determines a collaboration score associated with all computing devices in the computing system using a code segment. In some cases, the operations of this step may refer to, or be performed by, a code segment as described with reference to FIG. 9.

FIG. 12 shows an example of a process for determining collaboration scores in accordance with aspects of the present disclosure. In some examples, these operations may be performed by a system including a processor executing a set of codes to control functional elements of an apparatus. Additionally or alternatively, the processes may be performed using special-purpose hardware. Generally, these operations may be performed according to the methods and processes described in accordance with aspects of the present disclosure. For example, the operations may be composed of various substeps, or may be performed in conjunction with other operations described herein.

At step 1200, an electronic directory is provided for the storing and modifying of data elements. In some cases, the operations of this step may refer to, or be performed by, a data storage repository as described with reference to FIG. 9.

At step 1205, the system categorizes contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified. In some cases, the operations of this step may refer to, or be performed by, a first computing device as described with reference to FIG. 8.

At step 1210, the system modifies said individual data element in the directory in response to a request to modify by either (a) a contributor with an owner identifier as defined by the owner definition, (b) a set of informed contributors as defined by the informed definition, in excess of a threshold of informed contributors given in the informed definition, and (c) a set of uninformed contributors as defined by the uninformed definition, in excess of a threshold of uninformed contributors given in the uninformed definition. In some cases, the operations of this step may refer to, or be performed by, a data element as described with reference to FIGS. 9 and 10.

At step 1215, the system creates a collaboration data set associated with the contributor, this data set including a collaboration score determined as a function of at least three of the group of collaboration score factors including: response timeous, notification timeous, accuracy, engagement, and contactability, where each of at least three of the group of factors is weighted relative to one another. In some cases, the operations of this step may refer to, or be performed by, a collaboration data set as described with reference to FIG. 9.

At step 1220, the system determines a collaboration score associated with the contributor using a code segment. In some cases, the operations of this step may refer to, or be performed by, a code segment as described with reference to FIG. 9.

Accordingly, the present disclosure describes at least the following embodiments.

An apparatus for healthcare provider scoring is described. The apparatus may include a first computing device, the first computing device associated with an owner identifier, a second computing device, a data storage repository, a data records, a first data field, a first data element, a first owner identifier, a first collaboration data set associated with the first owner identifier, the first collaboration data set comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another, and a code segment for determining a collaboration score associated with the owner identifier, wherein the collaboration score is a function of the first collaboration data set.

Some examples may further include a second data field. Some examples may further include a second data element. Some examples may further include a second owner identifier. Some examples may further include a second collaboration data set associated with the second owner identifier, the second collaboration data set comprising a second collaboration score being determined as a function of a second at least three of the group of collaboration score factors comprising: response timeous, notification timeous, accuracy, engagement, and contactability, wherein each of the second at least three of the group of collaboration score factors is weighted relative to one another. In some examples, said code segment for determining said collaboration score associated with said owner identifier, wherein said collaboration score is a function of said first collaboration data set and the second collaboration data set.

Some examples of the apparatus, system, and method described above may further include a third computing device, the third computing device associated with a first informed user identifier. In some examples, the first data field. In some examples, the first informed user identifier. Some examples may further include a third collaboration data set associated with the first informed user identifier, the third collaboration data set comprising a third collaboration score being determined as a function of a third at least three of the group of collaboration score factors comprising: response timeous, notification timeous, accuracy, engagement, and contactability, wherein each of the third at least three of the group of collaboration score factors is weighted relative to one another. In some examples, the first informed user identifier.

Some examples may further include a fourth collaboration data set associated with the first informed user identifier, the fourth collaboration data set comprising a fourth collaboration score being determined as a function of a fourth at least three of the group of collaboration score factors comprising: response timeous, notification timeous, accuracy, engagement, and contactability, wherein each of the fourth at least three of the group of collaboration score factors is weighted relative to one another.

Some examples may further include a fourth computing device, the fourth computing device associated with a second informed user identifier. In some examples, the first data field. In some examples, the second informed user identifier. Some examples may further include a fifth collaboration data set associated with the second informed user identifier, the fifth collaboration data set comprising a fifth collaboration score being determined as a function of a fifth at least three of the group of collaboration score factors comprising: response timeous, notification timeous, accuracy, engagement, and contactability, wherein each of the fifth at least three of the group of collaboration score factors is weighted relative to one another. In some examples, the second informed user identifier.

Some examples may further include a sixth collaboration data set associated with the second informed user identifier, the sixth collaboration data set comprising a sixth collaboration score being determined as a function of a sixth at least three of the group of collaboration score factors comprising: response timeous, notification timeous, accuracy, engagement, and contactability, wherein each of the sixth at least three of the group of collaboration score factors is weighted relative to one another.

Some examples of the apparatus, system, and method described above may further include a fifth computing device, the fifth computing device associated with an uninformed user identifier. In some examples, the first data field. In some examples, the uninformed user identifier. In some examples, the first data field does not comprise a seventh collaboration data set associated with the uninformed user identifier. In some examples, the second data field. In some examples, the uninformed user identifier. In some examples, the second data field does not comprise an eighth collaboration data set associated with the uninformed user identifier.

In some examples, said first owner identifier identifies a first owner. In some examples, said second owner identifier identifies a second owner. In some examples, said first collaboration score is determined as a function of an age of each of the first at least three of the group of collaboration score factors. In some examples, said second collaboration score is determined as a function of an age of each of the second at least three of the group of collaboration score factors.

In some examples, said third collaboration score is determined as a function of an age of each of the third at least three of the group of collaboration score factors. In some examples, said fourth collaboration score is determined as a function of an age of each of the fourth at least three of the group of collaboration score factors. In some examples, said fifth collaboration score is determined as a function of an age of each of the fifth at least three of the group of collaboration score factors.

In some examples, said first collaboration score is determined as a function of an age of each of the first at least three of the group of collaboration score factors, wherein older ones of the first at least three of the group of collaboration score factors are weighted less than newer ones of the first at least three of the group of collaboration score factors.

In some examples, said second collaboration score is determined as a function of an age of each of the second at least three of the group of collaboration score factors, wherein older ones of the second at least three of the group of collaboration score factors are weighted less than newer ones of the second at least three of the group of collaboration score factors.

In some examples, said third collaboration score is determined as a function of an age of each of the third at least three of the group of collaboration score factors, wherein older ones of the third at least three of the group of collaboration score factors are weighted less than newer ones of the third at least three of the group of collaboration score factors.

In some examples, said fourth collaboration score is determined as a function of an age of each of the fourth at least three of the group of collaboration score factors, wherein older ones of the fourth at least three of the group of collaboration score factors are weighted less than newer ones of the fourth at least three of the group of collaboration score factors.

In some examples, said fifth collaboration score is determined as a function of an age of each of the fifth at least three of the group of collaboration score factors, wherein older ones of the fifth at least three of the group of collaboration score factors are weighted less than newer ones of the fifth at least three of the group of collaboration score factors.

An apparatus for healthcare provider scoring is described. The apparatus may include a computing device, a data storage repository, data records, a first data field, a first data element, an owner identifier, a first collaboration data set associated with the owner identifier, the first collaboration data set comprising a first collaboration score, the first collaboration score being determined as a function of a first at least three of a group of collaboration score factors comprising: response timeous, notification timeous, accuracy, engagement, and contactability, wherein each of the first at least three of the group of collaboration score factors is weighted relative to one another, and a code segment for determining a collaboration score associated with the owner identifier, wherein the collaboration score is a function of the first collaboration data set.

Some examples may further include a second data field. Some examples may further include a second data element. In some examples, the owner identifier. Some examples may further include a second collaboration data set associated with the owner identifier, the second collaboration data set comprising a second collaboration score being determined as a function of a second at least three of the group of collaboration score factors comprising: response timeous, notification timeous, accuracy, engagement, and contactability, wherein each of the second at least three of the group of collaboration score factors is weighted relative to one another. In some examples, said code segment for determining said collaboration score associated with said owner identifier, wherein said collaboration score is a function of said first collaboration data set and the second collaboration data set.

An apparatus for healthcare provider scoring is described. The apparatus may include a first data element, a first data cohort definition, the first data cohort definition comprising a first owner definition, a first informed cohort definition, and first uninformed cohort definition, and a first data item, wherein the first data item is configured to change in response to a first change request from at least one of (a) a first data owner defined by the first owner definition, (b) a plurality of informed requestors defined by the first informed cohort definition numbering in excess of a first informed cohort threshold defined by the first informed cohort definition, and (c) a plurality of uninformed requestors defined by the first uninformed cohort definition numbering in excess of a first uninformed cohort threshold defined by the first uninformed cohort definition.

Some examples of the apparatus, system, and method described above may further include a second data element. Some examples may further include a second data cohort definition, the second data cohort definition comprising a second owner definition, a second informed cohort definition, and a second uninformed cohort definition. Some examples may further include a second data item, wherein the second data item is configured to change in response to a second change request from at least one of (a) a second data owner defined by the second owner definition, (b) a plurality of informed requestors defined by the second informed cohort definition numbering in excess of a second informed cohort threshold defined by the second informed cohort definition, and (c) a plurality of uninformed requestors defined by the second uninformed cohort definition numbering in excess of a second uninformed cohort threshold defined by the second uninformed cohort definition.

A method for healthcare provider scoring is described. The method may include providing a computer system, comprising a number of computing devices with access to a data storage repository comprising data records with distinct data elements, categorizing computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements, modifying a data element in response to a request to modify by either (a) a computing device with an owner identifier as defined by the owner definition, (b) a plurality of informed computing devices as defined by the informed definition, in excess of a threshold of informed computing devices given in the informed definition, and (c) a plurality of uninformed computing devices as defined by the uninformed definition, in excess of a threshold of uninformed computing devices given in the uninformed definition, creating a collaboration data set associated with the identifier of an individual computing device, this data set comprising of at least three of the group of collaboration score factors comprising: response timeous, notification timeous, accuracy, engagement, and contactability, determining the collaboration score associated with the identifier of the computing device as a function of the collaboration data set, wherein each of at least three of the group of factors in the data sets are weighted relative to one another, using a code segment, and determining further a collaboration score associated with all computing devices in the computing system using a code segment.

An apparatus for a healthcare provider scoring system method in which to measure a provider's behavior is described. The apparatus may include a processor, memory in electronic communication with the processor, and instructions stored in the memory. The instructions may be operable to provide a computer system, comprising a number of computing devices with access to a data storage repository comprising data records with distinct data elements, categorize computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements, modify a data element in response to a request to modify by either (a) a computing device with an owner identifier as defined by the owner definition, (b) a plurality of informed computing devices as defined by the informed definition, in excess of a threshold of informed computing devices given in the informed definition, and (c) a plurality of uninformed computing devices as defined by the uninformed definition, in excess of a threshold of uninformed computing devices given in the uninformed definition, create a collaboration data set associated with the identifier of an individual computing device, this data set comprising of at least three of the group of collaboration score factors comprising: response timeous, notification timeous, accuracy, engagement, and contactability, determine the collaboration score associated with the identifier of the computing device as a function of the collaboration data set, wherein each of at least three of the group of factors in the data sets are weighted relative to one another, using a code segment, and determine further a collaboration score associated with all computing devices in the computing system using a code segment.

A non-transitory computer readable medium storing code for a healthcare provider scoring system method in which to measure a provider's behavior is described. In some examples, the code comprises instructions executable by a processor to: provide a computer system, comprising a number of computing devices with access to a data storage repository comprising data records with distinct data elements, categorize computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements, modify a data element in response to a request to modify by either (a) a computing device with an owner identifier as defined by the owner definition, (b) a plurality of informed computing devices as defined by the informed definition, in excess of a threshold of informed computing devices given in the informed definition, and (c) a plurality of uninformed computing devices as defined by the uninformed definition, in excess of a threshold of uninformed computing devices given in the uninformed definition, create a collaboration data set associated with the identifier of an individual computing device, this data set comprising of at least three of the group of collaboration score factors comprising: response timeous, notification timeous, accuracy, engagement, and contactability, determine the collaboration score associated with the identifier of the computing device as a function of the collaboration data set, wherein each of at least three of the group of factors in the data sets are weighted relative to one another, using a code segment, and determine further a collaboration score associated with all computing devices in the computing system using a code segment.

A system for healthcare provider scoring is described. The system may include means for providing a computer system, comprising a number of computing devices with access to a data storage repository comprising data records with distinct data elements, means for categorizing computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements, means for modifying a data element in response to a request to modify by either (a) a computing device with an owner identifier as defined by the owner definition, (b) a plurality of informed computing devices as defined by the informed definition, in excess of a threshold of informed computing devices given in the informed definition, and (c) a plurality of uninformed computing devices as defined by the uninformed definition, in excess of a threshold of uninformed computing devices given in the uninformed definition, means for creating a collaboration data set associated with the identifier of an individual computing device, this data set comprising of at least three of the group of collaboration score factors comprising: response timeous, notification timeous, accuracy, engagement, and contactability, means for determining the collaboration score associated with the identifier of the computing device as a function of the collaboration data set, wherein each of at least three of the group of factors in the data sets are weighted relative to one another, using a code segment, and means for determining further a collaboration score associated with all computing devices in the computing system using a code segment.

A method for healthcare provider scoring is described. The method may include providing an electronic directory for the storing and modifying of data elements, categorizing contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified, modifying said individual data element in the directory in response to a request to modify by either (a) a contributor with an owner identifier as defined by the owner definition, (b) a plurality of informed contributors as defined by the informed definition, in excess of a threshold of informed contributors given in the informed definition, and (c) a plurality of uninformed contributors as defined by the uninformed definition, in excess of a threshold of uninformed contributors given in the uninformed definition, creating a collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another, and determining a collaboration score associated with the contributor using a code segment.

An apparatus for a healthcare provider scoring system method in which to measure a provider's behavior is described. The apparatus may include a processor, memory in electronic communication with the processor, and instructions stored in the memory. The instructions may be operable to cause the processor to provide an electronic directory for the storing and modifying of data elements, categorize contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified, modify said individual data element in the directory in response to a request to modify by either (a) a contributor with an owner identifier as defined by the owner definition, (b) a plurality of informed contributors as defined by the informed definition, in excess of a threshold of informed contributors given in the informed definition, and (c) a plurality of uninformed contributors as defined by the uninformed definition, in excess of a threshold of uninformed contributors given in the uninformed definition, create a collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another, and determine a collaboration score associated with the contributor using a code segment.

A non-transitory computer readable medium storing code for a healthcare provider scoring system method in which to measure a provider's behavior is described. In some examples, the code comprises instructions executable by a processor to: provide an electronic directory for the storing and modifying of data elements, categorize contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified, modify said individual data element in the directory in response to a request to modify by either (a) a contributor with an owner identifier as defined by the owner definition, (b) a plurality of informed contributors as defined by the informed definition, in excess of a threshold of informed contributors given in the informed definition, and (c) a plurality of uninformed contributors as defined by the uninformed definition, in excess of a threshold of uninformed contributors given in the uninformed definition, create a collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another, and determine a collaboration score associated with the contributor using a code segment.

A system for healthcare provider scoring is described. The system may include means for providing an electronic directory for the storing and modifying of data elements, means for categorizing contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified, means for modifying said individual data element in the directory in response to a request to modify by either (a) a contributor with an owner identifier as defined by the owner definition, (b) a plurality of informed contributors as defined by the informed definition, in excess of a threshold of informed contributors given in the informed definition, and (c) a plurality of uninformed contributors as defined by the uninformed definition, in excess of a threshold of uninformed contributors given in the uninformed definition, means for creating a collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another, and means for determining a collaboration score associated with the contributor using a code segment.

Some examples of the method, apparatus, non-transitory computer readable medium, and system described above may further include providing an electronic directory for the storing and modifying of data elements. Some examples may further include categorizing a first contributor to a data element by an identifier tag, possible identifiers consisting of (a) an owner identifier, (b) an informed identifier, and (c) a uninformed identifier, with each of said identifiers being attributed to a first contributor by (a) a first owner identifier definition, (b) a first informed identifier definition, and (c) a first uninformed identifier definition, with each of the definitions being unique to the data element being modified. Some examples may further include modifying said individual data element in the directory in response to a request to modify by either (a) a first contributor with an owner identifier as defined by the owner definition, (b) a plurality of informed contributors as defined by the first informed definition, in excess of a threshold of informed contributors given in the first informed definition, and (c) a plurality of uninformed contributors as defined by the first uninformed definition, in excess of a threshold of first uninformed contributors given in the uninformed definition. Some examples may further include creating a first collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another. Some examples may further include determining a first collaboration score associated with the contributor using a code segment.

Some examples of the method, apparatus, non-transitory computer readable medium, and system described above may further include providing the same electronic directory for the storing and modifying of data elements. Some examples may further include categorizing a second contributor to a data element by an identifier tag, possible identifiers consisting of (a) an owner identifier, (b) an informed identifier, and (c) a uninformed identifier, with each of said identifiers being attributed to a second contributor by (a) a second owner identifier definition, (b) a second informed identifier definition, and (c) a second uninformed identifier definition, with each of the definitions being unique to the data element being modified. Some examples may further include modifying said individual data element in the directory in response to a request to modify by either (a) a second contributor with an owner identifier as defined by the owner definition, (b) a plurality of informed contributors as defined by the second informed definition, in excess of a threshold of informed contributors given in the second informed definition, and (c) a plurality of uninformed contributors as defined by the second uninformed definition, in excess of a threshold of second uninformed contributors given in the uninformed definition. Some examples may further include creating a second collaboration data set associated with the contributor, this data set comprising a collaboration score determined as a function of at least three of the group of collaboration score factors comprising: response timeous, notification timeous, accuracy, engagement, and contactability, wherein each of at least three of the group of factors is weighted relative to one another. Some examples may further include determining a collaboration score associated with the contributor using a code segment, wherein the collaboration score is a function of the first collaboration data set and the second collaboration data set.

Some of the functional units described in this specification have been depicted as labeled blocks, or components, to more particularly emphasize their implementation independence. For example, a block may be implemented as a hardware circuit comprising custom very large-scale integration (VLSI) circuits or gate arrays, off-the-shelf semiconductors such as logic chips, transistors, or other discrete components. A block may also be implemented in programmable hardware devices such as field programmable gate arrays, programmable array logic, programmable logic devices or the like.

Blocks may also be implemented in software for execution by various types of processors. An identified block of executable code may, for instance, comprise one or more physical or logical blocks of computer instructions that may, for instance, be organized as an object, procedure, or function. Nevertheless, the executables of an identified block need not be physically located together but may comprise disparate instructions stored in different locations which, when joined logically together, comprise the block and achieve the stated purpose for the block.

Indeed, a block of executable code could be a single instruction, or many instructions, and may even be distributed over several different code segments, among different programs, and across several memory devices. Similarly, operational data may be identified and illustrated herein within blocks and may be embodied in any suitable form and organized within any suitable type of data structure. The operational data may be collected as a single data set or may be distributed over different locations including over different storage devices, and may exist, at least partially, merely as electronic signals on a system or network.

While the invention herein disclosed has been described by means of specific embodiments, examples and applications thereof, numerous modifications and variations could be made thereto by those skilled in the art without departing from the scope of the invention set forth in the claims.

What is claimed is:

1. A system comprising:
a first computing device, the first computing device associated with an owner identifier;
a second computing device comprising:
a data storage repository comprising:
a data records comprising:
a first data field comprising:
a first data element;
a first owner identifier; and
a first collaboration data set associated with the first owner identifier, the first collaboration data set comprising a first collaboration score, the first collaboration score being determined as a function of a first at least three of a group of collaboration score factors comprising: response timeous, notification timeous, accuracy, engagement, and contactibility, wherein each of the first at least three of the group of collaboration score factors is weighted relative to one another; and
a code segment for determining a collaboration score associated with the owner identifier, wherein the collaboration score is a function of the first collaboration data set; and
a network, the network coupled to the first computing device and the second computing device.

2. The system of claim 1 further comprising:
said second computing device further comprising:
said data storage repository further comprising:
said data records further comprising:
a second data field comprising:
a second data element;
a second owner identifier; and
a second collaboration data set associated with the second owner identifier, the second collaboration data set comprising a second collaboration score being determined as a function of a second at least three of the group of collaboration score factors comprising: response timeous, notification timeous, accuracy, engagement, and contactibility, wherein each of the second at least three of the group of collaboration score factors is weighted relative to one another;
said code segment for determining said collaboration score associated with said owner identifier, wherein said collaboration score is a function of said first collaboration data set and the second collaboration data set.

3. The system of claim 2 further comprising:
a third computing device, the third computing device associated with a first informed user identifier;
the first data field further comprising:
the first informed user identifier;
a third collaboration data set associated with the first informed user identifier, the third collaboration data set comprising a third collaboration score being determined as a function of a third at least three of the group of collaboration score factors comprising: response timeous, notification timeous, accuracy, engagement, and contactibility, wherein each of the third at least three of the group of collaboration score factors is weighted relative to one another;
the second data field further comprising:
the first informed user identifier;
a fourth collaboration data set associated with the first informed user identifier, the fourth collaboration data set comprising a fourth collaboration score being determined as a function of a fourth at least three of the group of collaboration score factors comprising: response timeous, notification timeous, accuracy, engagement, and contactibility, wherein each of the fourth at least three of the group of collaboration score factors is weighted relative to one another;
a fourth computing device, the fourth computing device associated with a second informed user identifier;
the first data field further comprising:
the second informed user identifier;
a fifth collaboration data set associated with the second informed user identifier, the fifth collaboration data set comprising a fifth collaboration score being determined as a function of a fifth at least three of the group of collaboration score factors comprising: response timeous, notification timeous, accuracy, engagement, and contactibility, wherein each of the fifth at least three of the group of collaboration score factors is weighted relative to one another;
the second data field further comprising:
the second informed user identifier;
a sixth collaboration data set associated with the second informed user identifier, the sixth collaboration data set comprising a sixth collaboration score being determined as a function of a sixth at least three of the group of collaboration score factors comprising: response timeous, notification timeous, accuracy, engagement, and contactibility, wherein each of the sixth at least three of the group of collaboration score factors is weighted relative to one another.

4. The system of claim 3 further comprising:
a fifth computing device, the fifth computing device associated with an uninformed user identifier;
the first data field further comprising:
the uninformed user identifier;
wherein the first data field does not comprise a seventh collaboration data set associated with the uninformed user identifier;
the second data field further comprising:
the uninformed user identifier;
wherein the second data field does not comprise an eighth collaboration data set associated with the uninformed user identifier.

5. The system of claim 3 further comprising:
said first owner identifier identifying a first owner; and
said second owner identifier identifying a second owner.

6. The system of claim 3 further comprising:
said first collaboration score further being determined as a function of an age of each of the first at least three of the group of collaboration score factors;
said second collaboration score further being determined as a function of an age of each of the second at least three of the group of collaboration score factors;
said third collaboration score further being determined as a function of an age of each of the third at least three of the group of collaboration score factors;
said fourth collaboration score further being determined as a function of an age of each of the fourth at least three of the group of collaboration score factors; and
said fifth collaboration score further being determined as a function of an age of each of the fifth at least three of the group of collaboration score factors.

7. The system of claim 6 further comprising:
said first collaboration score further being determined as a function of an age of each of the first at least three of the group of collaboration score factors, wherein older ones of the first at least three of the group of collaboration score factors are weighted less than newer ones of the first at least three of the group of collaboration score factors;
said second collaboration score further being determined as a function of an age of each of the second at least three of the group of collaboration score factors, wherein older ones of the second at least three of the group of collaboration score factors are weighted less than newer ones of the second at least three of the group of collaboration score factors;
said third collaboration score further being determined as a function of an age of each of the third at least three of the group of collaboration score factors, wherein older ones of the third at least three of the group of collaboration score factors are weighted less than newer ones of the third at least three of the group of collaboration score factors;
said fourth collaboration score further being determined as a function of an age of each of the fourth at least three of the group of collaboration score factors, wherein older ones of the fourth at least three of the group of collaboration score factors are weighted less than newer ones of the fourth at least three of the group of collaboration score factors; and
said fifth collaboration score further being determined as a function of an age of each of the fifth at least three of the group of collaboration score factors, wherein older ones of the fifth at least three of the group of collaboration score factors are weighted less than newer ones of the fifth at least three of the group of collaboration score factors.

8. A system comprising:
a computing device comprising:
a data storage repository comprising:
data records comprising:
a first data field comprising:
a first data element;
an owner identifier; and
a first collaboration data set associated with the owner identifier, the first collaboration data set comprising a first collaboration score, the first collaboration score being determined as a function of a first at least three of a group of collaboration score factors comprising: response timeous, notification timeous, accuracy, engagement, and contactibility, wherein each of the first at least three of the group of collaboration score factors is weighted relative to one another;
a code segment for determining a collaboration score associated with the owner identifier, wherein the collaboration score is a function of the first collaboration data set.

9. The system of claim 8 further comprising:
said computing device further comprising:
said data storage repository further comprising:
said data records further comprising:
a second data field comprising:
a second data element;
the owner identifier; and
a second collaboration data set associated with the owner identifier, the second collaboration data set comprising a second collaboration score being determined as a function of a second at least three of the group of collaboration score factors comprising: response timeous, notification timeous, accuracy, engagement, and contactibility, wherein each of the second at least three of the group of collaboration score factors is weighted relative to one another;
said code segment for determining said collaboration score associated with said owner identifier, wherein said collaboration score is a function of said first collaboration data set and the second collaboration data set.

10. An electronic directory comprising:
a first data element comprising:
a first data cohort definition, the first data cohort definition comprising a first owner definition, a first informed cohort definition, and first uninformed cohort definition, and
a first data item, wherein the first data item is configured to change in response to a first change request from at least one of (a) a first data owner defined by the first owner definition, (b) a plurality of informed requestors defined by the first informed cohort definition numbering in excess of a first informed cohort threshold defined by the first informed cohort definition, and (c) a plurality of uninformed requestors defined by the first uninformed cohort definition numbering in excess of a first uninformed cohort threshold defined by the first uninformed cohort definition.

11. The electronic directory of claim 10 further comprising:
a second data element comprising:
a second data cohort definition, the second data cohort definition comprising a second owner definition, a second informed cohort definition, and a second uninformed cohort definition, and
a second data item, wherein the second data item is configured to change in response to a second change request from at least one of (a) a second data owner defined by the second owner definition, (b) a plurality of informed requestors defined by the second informed cohort definition numbering in excess of a second informed cohort threshold defined by the second informed cohort definition, and (c) a plurality of uninformed requestors defined by the second uninformed cohort definition numbering in excess of a second uninformed cohort threshold defined by the second uninformed cohort definition.

12. A method comprising:
providing a computer system, comprising a number of computing devices with access to a data storage repository comprising data records with distinct data elements;
categorizing computing devices by identifier tags, possible identifiers consisting of (a) an owner identifier, (b) an informed identifier, and (c) an uninformed identifier, with each of said identifiers being attributed to a computing device by (a) an owner identifier definition, (b) an informed identifier definition, and (c) an uninformed identifier definition, with each of the definitions being unique to the distinct data elements;
modifying a data element in response to a request to modify by either (a) a computing device with an owner identifier as defined by the owner definition, (b) a plurality of informed computing devices as defined by the informed definition, in excess of a threshold of informed computing devices given in the informed definition, and (c) a plurality of uninformed computing devices as defined by the uninformed definition, in excess of a threshold of uninformed computing devices given in the uninformed definition;
creating a collaboration data set associated with the identifier of an individual computing device, this data set comprising of at least three of the group of collaboration score factors comprising: response timeous, notification timeous, accuracy, engagement, and contactibility;
determining the collaboration score associated with the identifier of the computing device as a function of the collaboration data set, wherein each of at least three of the group of factors in the data sets are weighted relative to one another, using a code segment;
determining further a collaboration score associated with all computing devices in the computing system using a code segment.

13. A method comprising:
providing an electronic directory for the storing and modifying of data elements;
categorizing contributors to a data element by identifier tags, possible identifiers consisting of (a) an owner identifier, (b) an informed identifier, and (c) an uninformed identifier, with each of said identifiers being attributed to a contributor by (a) an owner identifier definition, (b) an informed identifier definition, and (c) an uninformed identifier definition, with each of the definitions being unique to the data element being modified;
modifying said individual data element in the directory in response to a request to modify by either (a) a contributor with an owner identifier as defined by the owner definition, (b) a plurality of informed contributors as defined by the informed definition, in excess of a threshold of informed contributors given in the informed definition, and (c) a plurality of uninformed contributors as defined by the uninformed definition, in excess of a threshold of uninformed contributors given in the uninformed definition;
creating a collaboration data set associated with the contributor, this data set comprising a collaboration score determined as a function of at least three of the group of collaboration score factors comprising: response timeous, notification timeous, accuracy, engagement, and contactibility, wherein each of at least three of the group of factors is weighted relative to one another; and
determining a collaboration score associated with the contributor using a code segment.
